(19) United States
(12) Patent Application Publication
(10) Pub. No.: US 2016/0271155 A1
(43) Pub. Date: Sep. 22, 2016

(54) Vitamin Formulations

(71) Applicants: NESTEC S.A., Vevey (CH); BOARD OF SUPERVISORS OF LOUISIANA STATE UNIVERSITY AND AGRICULTURAL AND MECHANICAL COLLEGE, Baton Rouge, LA (US)

(72) Inventors: Zhijun Liu, Baton Rouge, LA (US); Juan A. Ruiz, Covington, LA (US); Richard Copp, Covington, LA (US); Harold Koch, JR., Covington, LA (US)

(21) Appl. No.: 15/037,960

(22) PCT Filed: Nov. 20, 2014
(86) PCT No.: PCT/US2014/066715

§ 371 Date: May 19, 2016

Related U.S. Application Data
(60) Provisional application No. 61/907,275, filed on Nov. 21, 2013.

(51) Int. Cl. A61K 31/704; A61K 9/00; A61K 31/4415; A61K 31/198; A61K 31/519; A61K 31/714

(52) U.S. Cl. CPC A61K 31/704; A23L 1/302; A23L 1/2366; A23L 1/30; A23L 1/3002; A61K 31/519; A61K 31/714; A61K 31/4415; A61K 31/198; A61K 9/0053; A23V 2002/00

(57) ABSTRACT

The present invention provides formulations for enhancing bioavailability of watersoluble compounds including water-soluble vitamins. The formulations include a watersoluble vitamin and a glycoside selected from a diterpene glycoside and a triterpene glycoside. Methods for increasing the bioavailability of a water-soluble vitamin are also described.

### CROSS-REFERENCES TO RELATED APPLICATIONS

The present application claims priority to U.S. Provisional Patent Application No. 61/907,275, filed. Nov. 21, 2013, the teachings of which are hereby incorporated by reference in their entirety for all purposes.

### BACKGROUND OF THE INVENTION

The present invention relates to compositions and methods to enhance the bioavailability of water soluble substances such as water soluble vitamins. Examples of water-soluble vitamins include the B-vitamins, and more particularly, vitamin B₆, vitamin B₉, and vitamin B₁₂, or any combination thereof.

Bioavailability is the dose of the drug that is absorbed from the site of administration that ultimately reaches the systemic circulation. This parameter defines how much drug needs to be administered to reach its therapeutic level. The formulation of the drug can affect its bioavailability. For example, oral bioavailability depends in part on the dissolution of the active ingredient from a tablet or dosage form. Oral bioavailability also depends on the diffusion of the active ingredient through an aqueous environment. Despite being water soluble, it is important to maximize a substance's bioavailability.

Vitamin B₉ is involved in maturation of red blood cells and the synthesis of purines and pyrimidines which are required for development of the fetal nervous system. Adequate folic acid intake before conception and throughout the first trimester of pregnancy helps prevent certain brain and spinal cord defects such as spina bifida. Folate is absorbed in the duodenum and upper jejunum. The US recommended daily dose for folate is 400 μg and the upper limit is 1000 μg. Folate is essentially nontoxic. Deficiency produces megaloblastic anemia indistinguishable from that due to vitamin B₁₂ deficiency. A deficiency of folate in old age significantly increases the risk of developing dementia.

In view of the foregoing, there is a need in the art to effectively maximize the bioavailability of water soluble substances and especially water soluble vitamins. The present invention satisfies these and other needs.

### BRIEF SUMMARY OF THE INVENTION

The present invention relates to compositions and methods for improving absorption and bioavailability. More particularly, the present invention relates to compositions of a biologically active substance such as a vitamin together with a glycoside to increase the extent to which the substance is absorbed into the bloodstream of a subject after administration.

In one embodiment, the present invention provides a formulation for enhancing bioavailability of vitamin B₉, the formulation comprising, consisting essentially of, or consisting of:

vitamin B₉; and

a glycoside selected from the group of a diterpene glycoside or a triterpene glycoside.

In certain aspects, the vitamin B₉ is 1,5-methyl-tetrahydrofolate or a salt thereof. In certain aspects, the vitamin B₉ is a calcium salt of L-5-methyl-tetrahydrofolate. In certain aspects, the glycoside is a diterpene glycoside.

In another embodiment, the present invention provides a method for making a vitamin B₉ formulation the method comprising, consisting essentially of, or consisting of:
admixing vitamin B₉ and a glycoside selected from the group consisting of a diterpene glycoside and a triterpene glycoside in a solvent to form a vitamin B₉ admixture;
heating the vitamin B₉ admixture to form a clear vitamin B9 admixture; and
removing the solvent from the clear vitamin B₉ admixture to form the formulation of vitamin B₉.

In yet another embodiment, the present invention provides a method for increasing the bioavailability of vitamin B₉, the method comprising, consisting essentially of, or consisting of:
administering a formulation comprising vitamin B₉; and a glycoside selected from the group consisting of a diterpene glycoside and a triterpene glycoside, thereby increasing the bioavailability of vitamin B₉.

These and other aspects, objects and advantages will become more apparent when read with the detailed description of the invention and the drawings which follow.

### BRIEF DESCRIPTION OF THE DRAWINGS

FIG. 1A shows the structure of rubusoside. FIG. 1B shows the structure of stevioside. FIG. 1C shows the structure of rebaudioside A.

FIG. 2A and FIG. 2B show the structures of several diterpene glycosides isolated from Rubus or Stevia plants.

FIG. 3 shows the concentration of API (vitamin B₉) in rat plasma over time after oral gavage with baseline normalized for all treatments. Rats (n=8 per group) were orally administered at a dose equivalent to 19 mg/70 kg person in free form (1:0), or a formulated form of 1:4; 1:10 and 1:20. Vertical bars represent one standard error of the means (n=8).

### DETAILED DESCRIPTION OF THE INVENTION

### I. Definitions

As used herein, the term “formulation” includes compositions containing a biologically active substance such as a vitamin together with a glycoside. The formulation can include one or more pharmaceutically acceptable excipients.

As used herein, the term “bioavailability” includes the extent to which a substance is absorbed into the bloodstream of a subject after administration of a pharmaceutical formulation, and the amount of the substance that reaches the general circulation of the subject.

As used herein, the terms “enhancing bioavailability” and “increasing bioavailability” include administering a substance, such as a vitamin, so as to raise the bioavailability of the substance above the level at which it would be normally available. Administering the substance can include formulating the substance so as to increase the bioavailability. The substance can be formulated to increase the bioavailability by any suitable amount. In general, the methods of the present invention lead to bioavailability increases of at least about 10%, as compared to administration via control methods. Bioavailability levels can be determined by any suitable method, including analysis of the drug in a blood, plasma, serum, or urine sample taken from a subject after administration. Bioavailability can be assessed, for example, by plotting the concentration of a substance in the circulation of a subject over time after administration. Bioavailability can be considered in terms of the maximum (peak) concentration of the substance in the blood after administration, as well as in terms of the time required for the concentration of the substance to reach the peak concentration. The “area under the curve” (AUC) of the concentration-vs.-time plot can be calculated and used to determine the total amount of the substance that is absorbed into the blood stream after administration of a single dose.

As used herein, the term “water-soluble vitamin” includes vitamin C or a B vitamin.

As used herein, the term “B vitamin” includes thiamine (vitamin B₁), riboflavin, niacin/nicotinic acid/nicotinamide (vitamin B₃), folic acid, folinic acid, L-methylfolate, or L-5 methylfolate (vitamin B₉), pyridoxine/pyridoxal/pyridoxamine (vitamin B₆), biotin (vitamin B₇), pantothenic acid (vitamin B₅), and vitamin B₁₂.

As used herein, the term “vitamin B₆” includes pyridioxine, pyridoxal, pyridoxamine, and pharmaceutically acceptable salts thereof.

As used herein, the term “vitamin B₉” can include numerous forms. In a specific aspect, vitamin B₉ may be included in the form of folic acid. In another aspect, vitamin B₉ may be included one or more of the forms of folic acid, folacin, metafolin, folate and/or one or more natural isomers of folate including (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof and the salts and esters thereof. Vitamin B₉ may be in the form of a folate or folate derivative thereof that is eventually converted to 5-methyl-tetrahydrofolic acid in the body and/or is absorbed into the bloodstream as 5-methyl-tetrahydrofolic acid. Folates, such as folic acid and folate, are eventually absorbed in the body and converted to L-5-methyl-tetrahydrofolic acid. In another embodiment, vitamin B₉ may be in the form of a folate or folate derivative thereof that increases blood folate levels, thereby reducing homocysteine levels.

As used herein, the term “vitamin C” includes L-ascorbic acid, also known as (R)-3,4-dihydroxy-5-((S)-1,2-dihydroxyethyl)furan-2(5H)-one, and pharmaceutically acceptable salts thereof.

As used herein, the term “terpene” includes an organic compound having one or more isoprene-derived subunits. Terpenes are generally synthesized chemically or biochemically from isoprene (2-methyl-1,3-butadiene having the formula CH₂C(CH₃)CHCH₂, i.e., C₅H₈) and isoprene derivatives. A “monoterpene” is generally understood to contain two isoprene subunits and has a base molecular formula of C₁₀H₁₆. “Diterpenes” and “triterpenes” typically contain four and six isoprene subunits, respectively. Terpenes, including diterpenes and triterpenes, can contain isoprene subunits arranged in a linear or cyclic configuration. The linear or cyclic backbones can be substituted with one or more moieties including, but not limited to, hydroxy, oxo, and carboxy groups.

As used herein, the term “glycoside” includes a compound having one or more sugar moieties and a non-sugar moiety. The sugar moieties generally contain from 1 to 6 monosaccharide subunits having from 5 to 6 carbon atoms. Examples of typical monosaccharide subunits include, but are not limited to, glucose, allose, altrose, mannose, gulose, idose, galactose, talose, psicose, fructose, sorbose, tagatose, arabinose, lyxose, ribose, xylose, ribulose, and xylulose. The monosaccharide subunits can also be deoxy sugars, amino sugars, or sulfosugars. The monosaccharide subunits can be linked to each other in a number of configurations. For example, linkages can occur between the 1-carbon (the anomeric carbon) and the 4-carbon of adjacent monosaccharide subunits (i.e., a 1-4 linkage), the 1-carbon and the 3-carbon of adjacent monosaccharide subunits (i.e., a 1-3 linkage), the 1-carbon and the 6-carbon of adjacent monosaccharide subunits (i.e., a 1-6 linkage), or the 1-carbon and the 2-carbon of adjacent monosaccharide subunits (i.e., a 1-2 linkage). A monosaccharide subunit can be linked within a sugar moiety such that the anomeric carbon is in the α- or β-configuration. The sugar moieties can also include linkages between carbon atoms other than the 1-, 2-, 3-, 4-, and 6-carbons. The non-sugar moiety in a glycoside (“the aglycone”) is typically connected to a sugar moiety via an ether linkage.

As used herein, the term “diterpene glycoside” includes glycosides as defined above, wherein the non-sugar moiety is a diterpene. Examples of diterpene glycosides include, but are not limited to, rebaudiosides, suaviosides, goshonosides, paniculosides, stevioside, and rubososide. A stevioside is preferred.

As used herein, the term “triterpene glycoside” includes glycosides as defined above, wherein the non-sugar moiety is a triterpene. Examples of triterpene glycosides include, but are not limited to, abrusosides, cimiracemosides, lansiosides, leucospilotasides, frondoside A, eximisoside A, and quadranguloside.

As used herein, the term “stevioside” (CAS 57817-89-7) refers to (4α)-13-[(2-O-β-D-glucopyranosyl-β-D-glucopyranosyl)oxy]kaur-16-en-18-oic acid β-D-glucopyranosyl ester having the structure:

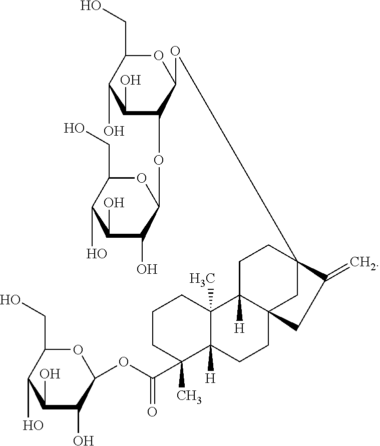

As used herein, the term “rebaudioside A” (CAS 58543-16-1) refers to (4α)-13-[(2-O-β-D-glucopyranosyl-3-O-β-Dglucopyranosyl-β-Dglucopyranosyl)-oxy]kaur-6-en-8-oic acid β-D-glucopyranosyl ester having the structure:

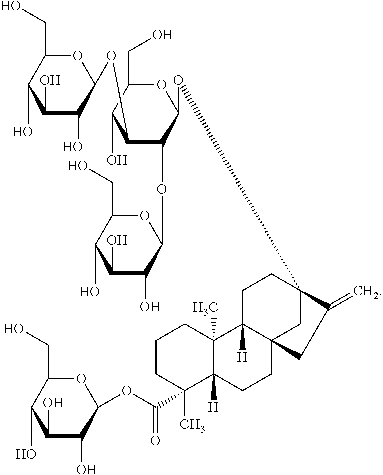

As used herein, the term “rebaudioside D” (CAS 63279-13-0) refers to (4α)-13-[(O-β-D-glucopyranosyl-(1→2)-O-[β-D-glucopyranosyl-(1→3)]-β-D-glucopyranosyl)oxy]kaur-16-en-18-oic acid 2-O-β-D-glucopyranosyl-β-D-glucopyranosyl ester having the structure:

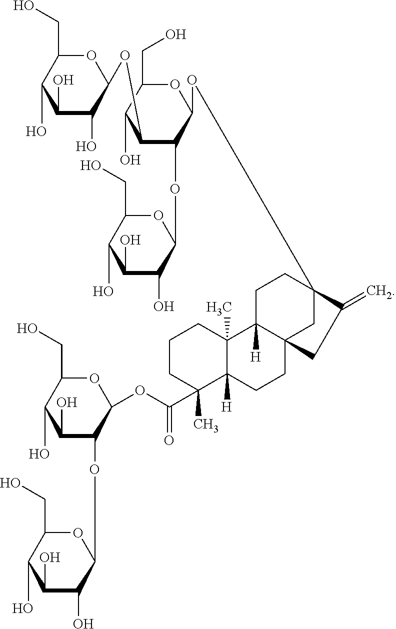

As used herein, the term “L-methylfolate” includes the compound (2S)-2-[[4-[(2-amino-5-methyl-4-oxo-1,6,7,8-tetrahydropteridin-6-yl)methylamino]benzoyl]amino]pentanedioic acid, having the CAS number 134-35-0, and pharmaceutically acceptable salts thereof.

Vitamin B₉ may be in the form of folate or reduced folates with various salts. In a specific embodiment, the folate and reduced folate are selected from the group consisting of D-glucosamine-folate, D-galactosamine-folate, D-glucosamine (6R,S)-tetrahydrofolate. D-glucosamine (6S)-tetrahydrofolate, D-glucosamine (6R)-tetrahydrofolate; D-galactosamine (6R,S)-tetrahydrofolate, D-galactosamine (6S)-tetrahydrofolate, D-galactosamine (6R)-tetrahydrofolate; D-glucosamine 5-methyl-(6R,S)-tetrahydrofolate, D-glucosamine 5-methyl-(6S)-tetrahydrofolate, D-glucosamine 5-methyl-(6R)-tetrahydrofolate; D-galactosamine 5-methyl-(6R,S)-tetrahydrofolate, D-galactosamine 5-methyl-(6S)-tetrahydrofolate, and D-galactosamine 5-methyl-(6R)-tetrahydrofolate.

As used herein, the term “N-acetylcysteine” includes the amino acid having the structure:

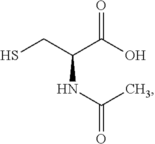

and pharmaceutically acceptable salts thereof.

### II. Embodiments

The present invention provides formulations and methods that increase the bioavailability of vitamin B₉ and other water-soluble compounds. The invention is based on the discovery that terpene glycosides can enhance the absorption of water-soluble compounds following oral administration. The formulations and methods can be used to administer a variety of other water-soluble compounds for use as nutritional supplements, nutraceuticals, and/or therapeutic agents.

### III. Formulations for Enhancing Bioavailability

The present invention provides a formulation for enhancing bioavailability. The formulation includes a water-soluble vitamin and a glycoside selected from a diterpene glycoside or a triterpene glycoside.

The present invention provides a formulation for enhancing bioavailability, wherein the water-soluble vitamin is a B vitamin. In some aspects, the B vitamin is selected from B₁. B₃, B₅, B₆, B₇, B₉, B₁₂, and a combination thereof. In some embodiments, the B vitamin is vitamin B₉. In some aspects, the formulation is substantially free of intrinsic factor. In some aspects, the water-soluble vitamin is vitamin C.

In one embodiment, the present invention provides a formulation for enhancing bioavailability of vitamin B₉, the formulation comprising:
vitamin B₉; and
a glycoside selected from the group consisting of a diterpene glycoside or a triterpene glycoside.

In certain aspects, vitamin B₉ is L-5-methyl-tetrahydrofolate or a salt thereof. Suitable salts include, for example, a calcium salt of L-5-methyl-tetrahydrofolate or a sodium salt of L-5-methyl-tetrahydrofolate.

In certain instances, the present invention provides a formulation, which comprises folate or folic acid, or a folic acid salt such as a pharmaceutically acceptable salt. In certain aspects, the formulation comprises folic acid, folinic acid, L-methylfolate, or L-5 methylfolate.

A. Terpene Glycosides

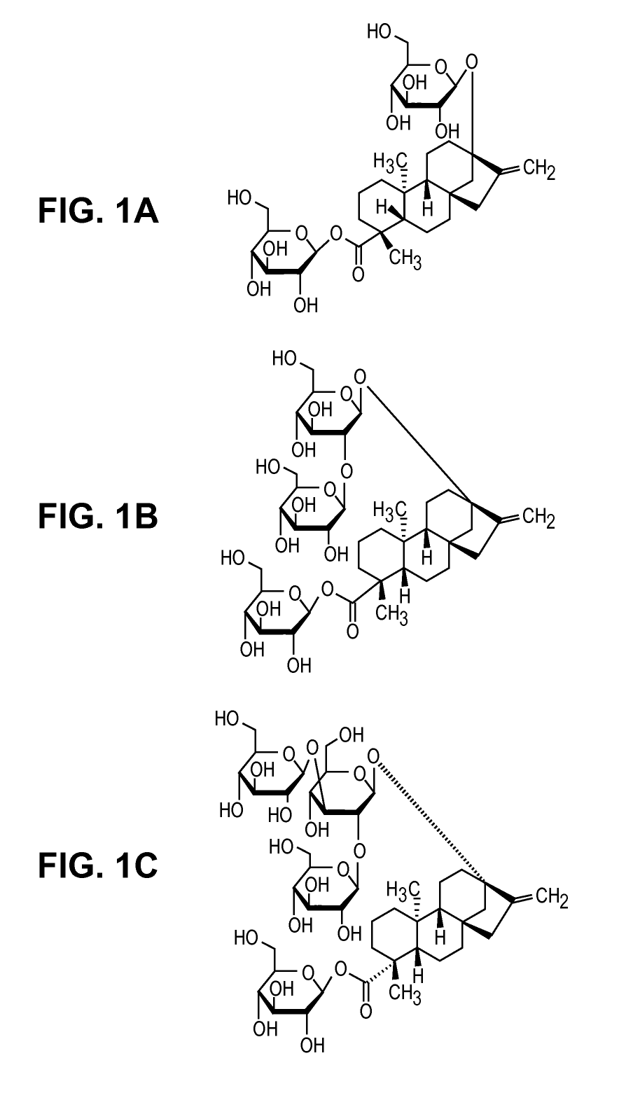

Natural terpene glycosides exist in a variety of plant sources. They generally are terpene aglycons attached to at least one glucose or other simple sugars (e.g., xylose or galactose), and the most common forms are monoterpene glycosides, diterpene glucosides, and triterpene glucosides. Many of these compounds are known to be non-toxic and natural sweeteners. Examples of diterpene glycosides include rubusoside, rebaudioside, stevioside, and steviol monoside. Rubusoside A is a diterpene glycoside mainly from Chinese sweet leaf tea leaves (Rubus suavissimus; Rosaceae). Rubusoside A has a molecular formula C₃₂H₅₀O₁₃ and molecular weight of 642.73. The structure of rubusoside is shown in FIG. 1A. Rubusoside also has good solubility in water, alcohol and acetone and ethyl acetate. The compound as shown in FIG. 1A is a diterpene aglycone with two glucose molecules attached.

Stevioside is a diterpene glycoside that is isolated from the Stevia leaf (Stevia rebaudiana; Asteraceae). Stevioside has a molecular formula C₃₈H₆₀O₁₈ and a molecular weight of 804. The structure is shown in FIG. 1B. The compound as shown is a diterpene aglycone with three glucose molecules. In pure form, it is a crystal or white powder. Another diterpene glycoside that is isolated from the Stevia leaf is rebaudioside, which exists in several forms, including rebaudioside A, rebaudioside B, rebaudioside C, rebaudioside D, rebaudioside E, and rebaudioside F. The structure of rebaudioside A is shown in FIG. 1C. The compound as shown is a diterpene aglycone with four glucose molecules. In pure form, it is a white powder.

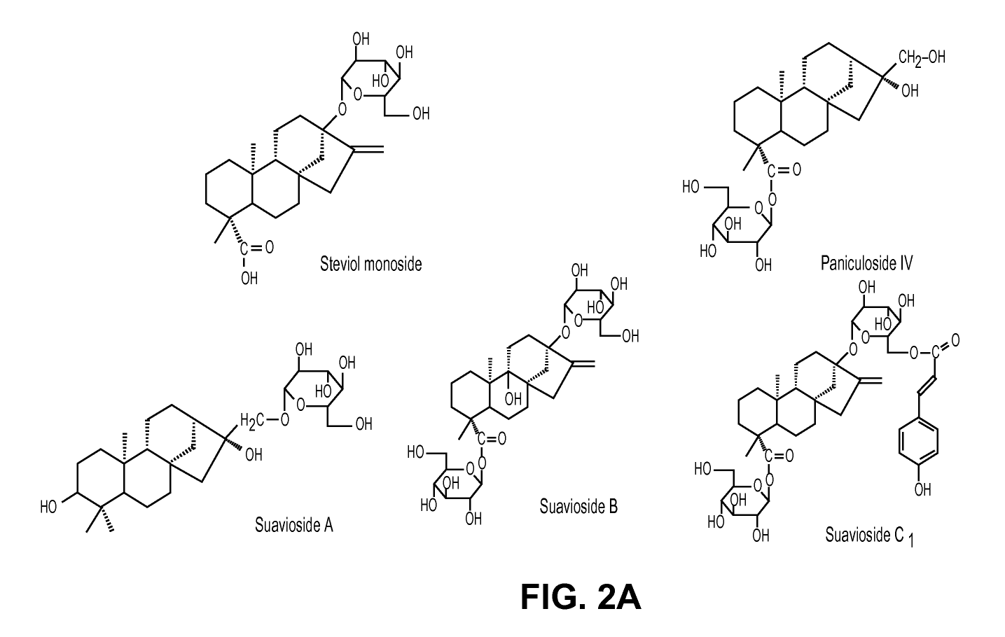

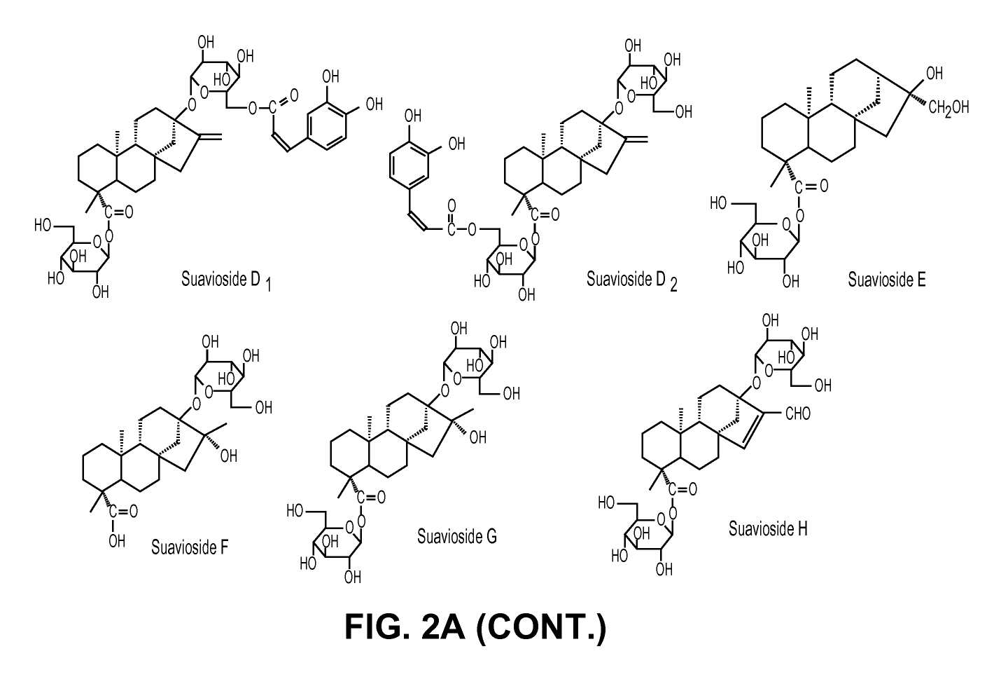

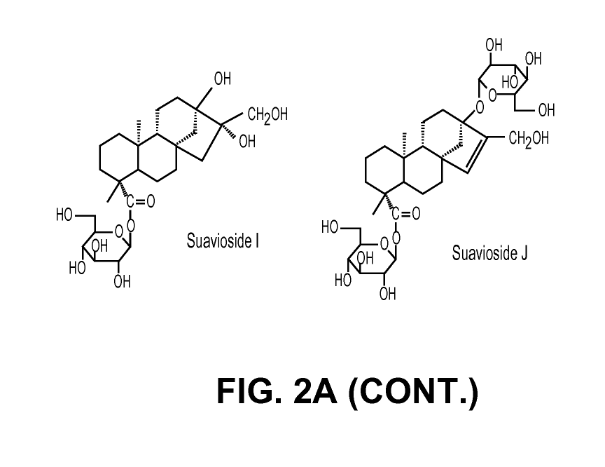

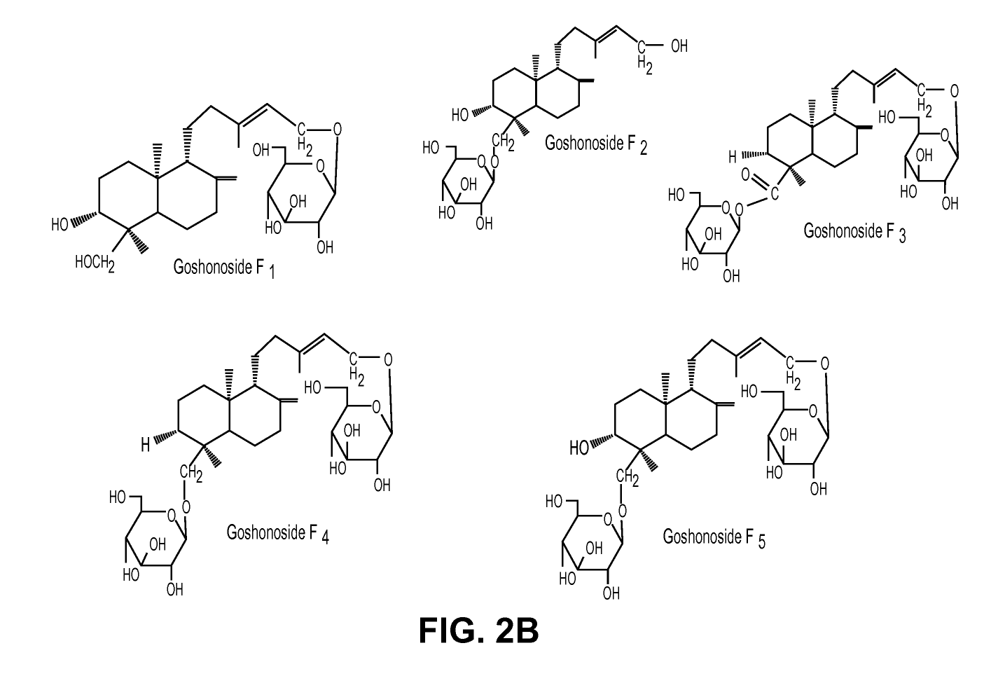

Another diterpene glycoside that is isolated from the Chinese sweet leaf tea (Rubus suavissimus; Rosaceae) and from stevia leaves (Stevia rebaudiana; Asteraceae) is steviol monoside. The structure of steviol monoside has only one glucose molecule (FIG. 2A) rather than two as in rubusoside. Steviol monoside can be isolated from the sweet leaf tea, stevia leaves, or be obtained through the partial acid or alkaline hydrolysis of rubusoside to cleave one glucose molecule. Unlike rubusoside, steviol monoside is not a dominant diterpene glycoside in the sweet leaf tea or stevia plant.

Other diterpenes that contain various numbers of glucose moieties are known. These compounds include: paniculoside IV, suaviosides A, B, C₁, D₁, D₂, E, F, G, H, I, and J (FIG. 2A) as identified by Ohtani et al. (1992, Phytochemistry 31(5): 1553-1559), and goshonosides F₁ to F₅ (FIG. 2B) as identified by Seto et al. (1984, Phytochemistry 23 (12): 2829-2834). Although many diterpene glycosides such as stevioside, rebaudioside A, rubusoside, steviol monoside, and suavioside B, G, I, J, and H taste sweet, other diterpene glycosides are tasteless or bitter. For example, paniculoside IV is tasteless, suavioside C₁ tastes bitter, suavioside D₁ is tasteless, suavioside D₂ tastes bitter, suavioside E is tasteless, and suavioside F tastes bitter as indicated by Ohtani et al. (1992, Phytochemistry 31(5): 1553-1559).

Accordingly, some embodiments of the present invention provide a formulation for enhancing bioavailability as described above, wherein the glycoside is a diterpene glycoside. In some embodiments, the diterpene glycoside is selected from rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5. In some embodiments, the diterpene glycoside is selected from stevioside, rebaudioside A, and rebaudioside D. In some embodiments, the diterpene glycoside is stevioside.

Any suitable water-soluble vitamin or other water-soluble compound can be combined with any suitable terpene glycoside to prepare a formulation of the invention. For example, vitamin C or a B vitamin can be combined with a diterpene glycoside or a triterpene glycoside. A B-vitamin can be combined with a rebaudioside, a suavioside, a goshonoside, rubusoside, or stevioside. Vitamin B₉ can be combined with a rebaudioside, a suavioside, a goshonoside, rubusoside, or stevioside. Vitamin C can be combined with a rebaudioside, a suavioside, a goshonoside, rubusoside, or stevioside. In some embodiments, the water-soluble vitamin is vitamin B₉ and the diterpene glycoside is stevioside. Other combinations of water-soluble compounds and terpene glycosides can be used in the formulations of the invention.

Any suitable amount of terpene glycoside can be used in formulations of the present invention. In general, the amount of terpene glycoside is sufficient to increase the bioavailability of the water-soluble vitamin or other water-soluble substance. The ratio of the water-soluble vitamin to the terpene glycoside is typically from about 10:1 to about 1:500 by weight. The ratio of the water-soluble vitamin to the terpene glycoside can be for example, about 10:1, about 5:1, about 1:1, about 1:4, about 1:6, about 1:8, about 1:10, about 1:15, about 1:20, about 1:25, about 1:30, about 1:35, about 1:40, or about 1:50 by weight. In some embodiments, the ratio of the water-soluble vitamin to the terpene glycoside is from about 1:1 to about 1:30 by weight. In some embodiments, the ratio of the water-soluble vitamin to the terpene glycoside is from about 1:10 to about 1:50 by weight. In some embodiments, the ratio of the water-soluble vitamin to the terpene glycoside is from about 1:25 by weight. In some embodiments, the ratio of the water-soluble vitamin to the terpene glycoside is from about 1:1 to about 1:0 by weight.

In certain aspects, the formulation has a ratio of vitamin B₉:to the glycoside of 1:1 w/w to 1:30 w/w. In other aspects, the formulation has a ratio of vitamin B₉:to the glycoside of 1:1 w/w to 1:25 w/w. In other aspects, the formulation has a ratio of vitamin B₉:to the glycoside of 1:1 w/w to 1:20 w/w. In certain aspects, the formulation has a ratio of vitamin B₉:to the glycoside of 1:10 w/w. In yet other aspects, ratio of vitamin B₉:to the glycoside is 1:10; 1:11; 1:12; 1:13; 1:14; 1:15; 1:16; 1:17; 1:18; 1:19; 1:20; 1:21; 1:22; 1:23; 1:24 or 1:25.

In certain instances, the formulation is a vitamin complex or a particle such as a nanoparticle. In certain aspects, the present invention provides vitamin B₉ and a glycoside, which forms a nanoparticle that is about 0.1 nm to about 10 nm in diameter. In other aspects, the vitamin B₉ and glycoside form a nanoparticle that is about 1 nm to about 5 nm in diameter, such as 1 nm, 2 nm, 3 nm, 4 nm, or 5 nm.

In certain instances, the complex or particle of vitamin and glycoside is held together by Van der Waals forces, with no covalent bond formation.

In certain instances, the formulation is a polydisperse population of nanoparticles ranging from 1 nm to about 10 nm in diameter or 1 nm to about 5 nm in diameter.

In certain instances, the stevioside: B₉ complex comprises a nanoparticle, or a nanomicelle, with a characteristic size distribution of approximately 2 nm to about 4 nanometers. This size, along with their physical and chemical properties, is believed to be important regarding their bioavailability enhancement properties. Nanoparticle size distribution can be measured using Dynamic Light Scattering (DLS).

The formulations of the present invention can contain water-soluble vitamins in combination with each other, as well as with other vitamins, nutrients, and drug compounds. Some embodiments of the invention provide a formulation including vitamin B₉ as described above, further including vitamins B₁₂ and B₆. In some embodiments, the formulation includes vitamin B₉, and further includes B₁₂ and N-acetylcysteine. In some embodiments, the formulation includes vitamin B₉, and further includes B₁₂.

A. Formulations

Depending on the intended mode of administration, the pharmaceutical compositions can be in the form of a solid, a semi-solid or liquid dosage form, preferably in unit dosage form suitable for administration of precise dosages. In addition to the water-soluble vitamin and the terpene glycoside, the compositions can contain pharmaceutically-acceptable excipients. “Pharmaceutically acceptable excipient” refers to an excipient or mixture of excipients which does not interfere with the biological activity and bioavailability of the vitamin(s) and which is not toxic or otherwise undesirable to the subject to which it is administered. Since acceptable carriers and excipients are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, such as sodium acetate, sorbitan monolaurate, triethanolamine acetate, and triethanolamine oleate. If desired, flavoring and/or coloring agents may be added as well. Other optional excipients for incorporation into an oral formulation include preservatives, suspending agents, thickening agents, and the like.

Accordingly, some embodiments of the invention provide a formulation for enhancing bioavailability as described above, wherein the formulation is in the form selected from the group consisting of a capsule, a tablet, a softgel, a powder, an effervescent form, or a lozenge.

Injectable formulations can be prepared in conventional forms, either as liquid solutions or suspensions, solid forms suitable for solubilization or suspension in liquid prior to injection, or as emulsions or liposomal formulations. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. Solutions can contain antioxidants, buffers, bacteriostats, and solutes that render the formulation isotonic with the blood of the intended recipient. Aqueous and non-aqueous sterile suspensions can include suspending agents, solubilizers, thickening agents, stabilizers, and preservatives. Sterile, fixed oils, fatty esters or polyols can be employed as non-aqueous solvents or suspending media. Injection solutions and suspensions can also be prepared from sterile powders, granules, and tablets.

Suitable formulations for rectal administration include, for example, suppositories, which includes an effective amount of a packaged composition with a suppository base. Suitable suppository bases include natural or synthetic triglycerides or paraffin hydrocarbons. In addition, it is also possible to use gelatin rectal capsules which contain a combination of the composition of choice with a base, including, for example, liquid triglycerides, polyethylene glycols, and paraffin hydrocarbons.

### IV. Methods for Making

In another embodiment, the present invention provides a method for making a formulation of vitamin B₉, the method comprising:
admixing vitamin B₉ and a glycoside selected from the group of a diterpene glycoside or a triterpene glycoside in a solvent to form a vitamin B₉ admixture;
heating the vitamin B₉ admixture to form a clear vitamin B₉ admixture; and
removing the solvent from the clear vitamin B₉ admixture to form the formulation of vitamin B₉.

In certain other aspects, the glycoside is a diterpene glycoside.

In certain aspects, the diterpene glycoside is a member selected from the group of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

In certain aspects, the diterpene glycoside is a member selected from the group of stevioside, rebaudioside A and rebaudioside D. Preferably, the diterpene glycoside is stevioside.

In certain aspects, the formulation has a ratio of vitamin B₉:to the glycoside of 1:1 w/w to 1:100 w/w. In other aspects, the formulation has a ratio of vitamin B₉:to the glycoside of 1:20 w/w to 1:50 w/w. The ratio of the water-soluble vitamin to the glycoside can be for example, about 10:1, about 5:1, about 1:1, about 1:4, about 1:6, about 1:8, about 1:10, about 1:15, about 1:20, about 1:25, about 1:30, about 1:35, about 1:40, or about 1:50 by weight. In certain other aspects, the formulation has a ratio of vitamin B₉:to the glycoside of 1:25 w/w to 1:30 w/w or 1:20 w/w to 1:30 w/w such as 1:20, 1:21, 1:22, 1:23, 1:24, 1:25, 1:26, 1:27, 1:28, 1:29, or 1:30 w/w.

In certain aspects, the solvent used in the methods of making is water. Water is the preferred solvent. In other aspects, water can be mixed with other solvents or other solvents can be used alone or mixed together. Suitable alcohols include, but are not limited to, methanol, ethanol, propanol, butanol, pentanol, hexanol, or a combination thereof. Other solvents include, but are not limited to, water, a C₃-C₈ ether, a C₃-C₆ ketone, a C₃-C₆ ester, or a mixture thereof.

Typically, the active ingredient or vitamin is added to a reaction flask and a solvent (e.g. water) is added. The ratio of active ingredient to solvent (e.g. water) is about 1:1 to about 1:500, or 1:100 to about 1:500, or about 1:150 to about 1:300 or 1:250. Thereafter, the glycoside is added. The reaction mixture is heated to about 40° C. to about 100° C., or about 50° C. to about 80° C., or about 60° C. to about 80° C. After heating from about 1 minute to about 60 minutes, vitamin B₉ and glycoside admixture form a clear vitamin B₉ admixture. Heating can be 1 minute, 5, 10, 15, 20, or 30 minutes. Removing the solvent (e.g., water) forms a dry powder. The water can be removed by lyophilization or spray drying.

In certain aspects, high shear mixing can be used which allows for minimal or no external heating. The high shear speeds up dissolution. The shear mixing generates heat and promotes dissolution.

In certain aspects, the powder of vitamin B₉ and the glycoside forms a nanoparticle. The nanoparticle has a size of about 0.1 nm to about 10 nm in diameter, or about 1 nm to about 5 nm in diameter.

A. Spay Drying

In general, spray drying methods employed herein produce a dry powder from a liquid suspension or slurry (e.g., aqueous) by rapidly drying with a hot gas or air. Typically, air or an inert gas is the heated drying medium, and an atomizer or spray nozzle is used to disperse the liquid suspension or slurry into a controlled drop size spray. Typically, the spray drying drop sizes ranges from about 1 nm to 500 μm. In certain instances, the complexes produced herein are in the 1 nm to 10 μm diameter range. In other instances, the spray drying can be 1 nm to 30 nm such as 1, 5, 10, 15, 20, 25, or 30 nm. The dry powder that is generated is free-flowing.

With respect to Vitamin B₉, the solvent used for the slurry is typically water. In other aspects, water can be used together with other solvents. In certain instances, the preferred solvent for spray drying Vitamin B₉ is water. With respect to Vitamin B₉, the preferred solvent for spray-drying is water.

### V. Methods for Increasing the Bioavailability of a Water-Soluble Vitamin

In another embodiment, the present invention provides a method for increasing the bioavailability of a water-soluble vitamin. The method includes administering a formulation containing a water-soluble vitamin and a glycoside selected from a diterpene glycoside and a triterpene glycoside, thereby increasing the bioavailability of the water-soluble vitamin.

The methods of the invention can include administering any of the formulations described above so as to increase the bioavailability of the water-soluble vitamin. The increase in the bioavailability can be observed by any suitable means, including as an increase in the area under the curve (AUC) for a plot of the vitamin's circulation concentration over time after administration of the formulation. Accordingly, some embodiments of the invention provide a method for increasing the bioavailability of a water-soluble vitamin, wherein the water-soluble vitamin has increased bioavailability (AUC) as a result of the formulation. As described above, the methods of the present invention generally lead to bioavailability increases of at least about 10%, as compared to administration via control methods. The bioavailability can be increased, for example, by 25%, or 50%, or 100% (i.e., by 1 fold). The bioavailability can be increased by 200% (i.e., by 2 fold). In some embodiments, the bioavailability of the water-soluble vitamin is increased between about 1 to 10 fold over a control. In some aspects, the bioavailability of the water-soluble vitamin is increased between about 2 to 6 fold over a control. The control methods typically include administration of vitamin formulations that do not contain terpene glycosides.

In certain instances, the bioavailability of vitamin B₉ is increased between about 1 to 10 such as 1, 2, 3, 4, 5, 6, 7, 8, 9, or 10 fold over a control. In other instances, the bioavailability of vitamin B₉ is increased between about 2 to 6 fold over a control.

Frequency of administration of the formulations described herein, as well as dosage, will vary from individual to individual, and may be readily established using standard techniques. A suitable dose is an amount of a formulation that, when administered as described above, is capable of providing a beneficial level of a water-soluble vitamin or other water-soluble compound in the circulation of the subject to whom the formulation is administered. Those of skill in the art are aware of the routine experimentation that will produce an appropriate dosage range for a subject in need of treatment. Those of skill are also aware that results provided by in vitro or in vivo experimental models can be used to extrapolate approximate dosages for a patient in need of treatment.

### Examples

### Example 1

### Preparation of Vitamin B₉ Formulations

Vitamin B₉ (5-methyl folate, MW 459.46) was formulated using stevioside to form a complex. In the Table 1 below, the vitamin to stevioside ratio was 1:0; 1:1; 1:4; 1:7 and 1:10.

Concentration ratios of 5-methylfolate (B₉) in stevioside and their resultant solution appearance is tabulated. All water solutions were prepared to contain 15 mg/mL B₉ and filtered before HPLC analysis.

TABLE 1

| | Concen- | | | | |
|---|---|---|---|---|---|
| | tration | | | | |
| | (mg/mL | | | | |
| | in | Solution | Particle Size | | |
| B9:ST | water) | Appearance | MV | MN | Note |

| | | | | | |
|---|---|---|---|---|---|
| 1:0 | 8.6 | Suspension | — | — | Intrinsic solubility, |
| | | | | | No homogenization |
| | | | | | or heat |
| 1:0 | 9.2 | Suspension | 1,917 | 1,865 | Blank control. |
| | | | | | Homogenization for |
| | | | | | 5 min and heat at |
| | | | | | 80° for 5 min |
| 1:1 | 9.7 | Suspension | 891 | 738 | Homogenization for |
| | | | | | 5 min and heat at |
| | | | | | 80° for 5 min |
| 1:4 | 11.2 | Suspension | 703 | 461 | Homogenization for |
| | | | | | 5 min and heat at |
| | | | | | 80° for 5 min |
| 1:7 | 12.5 | Clear, slight | 3.9 | 3.3 | Homogenization for |
| | | precipitation | | | 5 min and heat at |
| | | | | | 80° for 5 min |
| 1:10 | 15.3 | Completely | 3.4 | 3.0 | Homogenization for |
| | | clear | | | 5 min and heat at |
| | | | | | 80° for 5 min |

Concentrations of 5-methylfolate (B9) to stevioside at the 1:7 and 1:10 ratio produced particle sizes in the 3-4 nm size.

### Example 2

### Stability Studies of Reconstituted B₉ Formulations

Table 2 below shows stability data for reconstituted water solutions at various ratios of B₉ to stevioside.

| | | Weight | Water | Conc. of B9 | | |
|---|---|---|---|---|---|---|
| Products | Process | mg | mL | mg/mL | Solubility | Stability |
| B9-ST 1:1 | dry to powder | 30 | 0.97 | 15 | Completely | 5 h |
| | by Rotavap | | | | dissolved, Clear | |
| B9-ST 1:1 | dry to powder | 60 | 0.94 | 30 | Completely | 0.5 h |
| | by Rotavap | | | | dissolved, Clear | |
| B9-ST 1:4 | dry to powder | 75 | 0.94 | 15 | Completely | 3 h |
| | by Rotavap | | | | dissolved, Clear | |
| B9-ST 1:4 | dry to powder | 150 | 0.89 | 30 | Completely | 2 h |
| | by Rotavap | | | | dissolved, Clear | |
| B9-ST 1:7 | dry to powder | 120 | 0.89 | 15 | Completely | 1 h |
| | by Rotavap | | | | dissolved, Clear | |
| B9-ST 1:7 | dry to powder | 240 | 0.85 | 30 | Completely | 1 h |
| | by Rotavap | | | | dissolved, Clear | |
| B9-ST 1:10 | dry to powder | 165 | 0.89 | 15 | Completely | >6 h |
| | by Rotavap | | | | dissolved, Clear | |
| B9-ST 1:10 | dry to powder | 300 | 0.80 | 30 | Completely | 5 h |
| | by Rotavap | | | | dissolved, Clear | |

The solubility of 5-methylfolate (L-5-MTHF) free acid and Na₂ salt in different solvents is tabulated in Table 3.

TABLE 3

| | Concen- | | | |
|---|---|---|---|---|
| | tration | | | |
| | prepared | | | |
| Compound | (mg/mL) | Solvent | Solubility | Solubility |
| L-5-MTHF | 1.06 | Water | Suspension | <0.0016 mg/mL; |
| Free acid | 5.31 | Methanol/ | | below |
| | | Ethanol/ | | HPLC-UV detection |
| | | Acetone | | |
| | 8.09 | Oil/ | | |
| | | Petroleum | | |
| | | ether | | |
| L-5-MTHF | 56.53 | Water | Completely | >56.53 mg/mL |
| Na₂ Salt | | | clear | |

The results indicate that 5-methylfolate calcium salt has an intrinsic solubility in water of 8.6 mg/mL. The complex was processed in water. The API can be solubilized to 15 mg/mL or 30 mg/mL of water by forming complex with stevioside. The API/STV complex was dried to powder form (by rotavap) at various ratios of API:STV from 1:0 (control, no STV), 1:4, 1:10, and 1:20. Each complex powder is readily reconstituted fully and completely to a clear water solution with stability over time varying from 30 min to longer than 6 hours, depending on the ratio and API concentrations.

### Example 3

### Absorption Assessment in Caco-2 Cell Monolayer

A Caco-2 cellular model was used to study the effect of the stevioside on the intestinal absorption of vitamin B₉. The Caco-2 cell monolayer model is the standard for predicting human oral absorption. The test samples included: B₉:stevioside at 1:0; 1:4; 1:10 and 1:20.

Table 4 tabulates the permeability of methylfolate calcium (B₉) in Caco-2 monolayer cell culture.

TABLE 4

| | | | | Permeation |
|---|---|---|---|---|
| | | Assay duration | Pₐₚₚ (A-B) | enhancement (over |
| Formulation | Composition | (hr) | (10⁻⁶ · cm· s⁻¹) | 1:0) |
| B9-STV1:0 | 76 μg/mL B9 and 0 mg/mL STV | 2 | 5.89 ± 0.245 | 1.00 |
| B9-STV1:4 | 76 μg/mL B9 and 0.304 mg/mL STV | 2 | 7.94 ± 0.612 | 1.34 |
| B9-STV1:10 | 76 μg/mL B9 and 0.76 mg/mL STV | 2 | 5.97 ± 0.589 | 1.01 |
| B9-STV1:20 | 76 μg/mL B9 and 1.52 mg/mL STV | 2 | 12.21 ± 1.15 | 2.07 |

Using the Caco-2 cell monolayer assay, it was found that the vitamin B₉ calcium was permeable. A value of 6 predicts a 70-80% oral absorption. The use of stevioside at the ratio of 1:20 doubles the value to 12, which predicts a near 100% oral absorption. The use of stevioside at ratios of 1:4 to 1:10 had less of an effect on absorption than the ratio of 1:20.

### Example 4

### Absorption Assessment in a Rat Model

To assess whether the solubilizer enhanced the absorption of vitamin B₉, the four formulations were assessed in a Sprague Dawley rat model at the vitamin B₉ dose equivalent to 19 mg/70 kg person. Each group (formulation) had 8 rats (n=8). Each rat was orally gavaged and blood collected at 11 time points during a 12-hour period. The plasma samples were stored in a freezer prior to vitamin B₉ analyses.

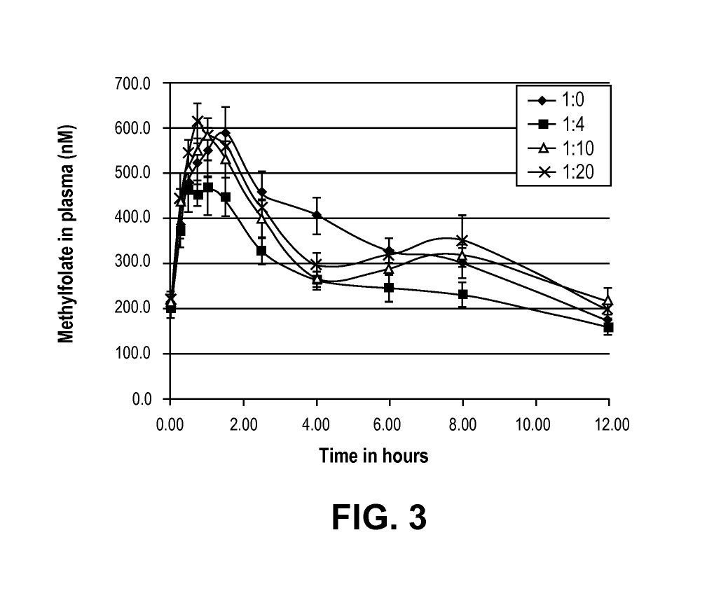

FIG. 3 shows the concentration of API (vitamin B₉) in rat plasma over time after oral gavage with baseline normalized for all treatments. Rats (n=8 per group) were orally administered at a dose equivalent to 19 mg/70 kg person in free form (1:0), or a formulated form of 1:4; 1:10 and 1:20. Vertical bars represent one standard error of the means (n=8).

Relative bioavailability was calculated by comparing the area under the time-concentration curves of formulated over the unformulated control. The results are tabulated in Table 5. The table contains various pharmacokinetic parameters in rats orally administered with various methylfolate Ca (B₉) formulations. Shown as Average±SD (% over the 1:0 control formulation).

TABLE 5

| Parameter | 1:0 | | 1:4 | | 1:10 | | 1:20 | |
|---|---|---|---|---|---|---|---|---|
| AUC_{0-∞} (ng min | 6476.06 ± 706.11 | (100) | 4958.15 ± 440.93 | (77) | 6828.80 ± 915.31 | (105) | 7126.93 ± 991.72 | (110) |
| mL⁻¹) | | | | | | | | |
| AUC⁰⁻¹² (ng min | 4159.49 ± 301.12 | (100) | 3245.82 ± 250.85 | (78) | 3938.85 ± 281.56 | (95) | 4131.49 ± 337.78 | (99) |
| mL⁻¹) | | | | | | | | |
| T_{1/2} (h) | 8.60 ± 1.92 | (100) | 7.31 ± 0.79 | (85) | 8.71 ± 1.11 | (101) | 9.84 ± 1.46 | (114) |
| MRT_{0-∞} (h) | 12.41 ± 2.40 | (100) | 11.03 ± 0.99 | (89) | 13.15 ± 1.46 | (106) | 13.92 ± 2.01 | (112) |
| CL (mL/h kg⁻¹) | 0.69 ± 0.06 | (100) | 0.89 ± 0.10 | (129) | 0.68 ± 0.08 | (99) | 0.65 ± 0.07 | (94) |
| Vss (mL kg⁻¹) | 7.70 ± 0.95 | (100) | 9.46 ± 0.77 | (123) | 8.22 ± 0.52 | (109) | 8.38 ± 0.94 | (106) |
| Cₘₐₓ (ng mL⁻¹) | 630.00 ± 49.55 | (100) | 539.71 ± 54.96 | (86) | 632.88 ± 46.15 | (100) | 649.33 ± 36.39 | (103) |
| Tₘₐₓ (h) | 1.59 ± 0.36 | (100) | 0.86 ± 0.19 | (56) | 0.91 ± 0.12 | (56) | 1.03 ± 0.13 | (63) |

Overall, the low ratio formulation (1:4) is below the control (1:0) in every PK parameters. The 1:10 ratio caught up with the control, and the highest ratio of 1:20 outperformed the control in several parameters. When predicted over an indefinite length of time, use of higher stevioside ratios of 1:10 and 1:20 led to 5% to 10% more bioavailability over the unformulated control. This is a reflection that stevioside complexed API stays in plasma at higher levels after the 12 hours (last blood collection). There are some interesting changes in the pharmacokinetic parameters related to the use of the high stevioside ratio of 1:20 over the control (1:0). These include the following: half-life was 14% longer; mean residence time was 12% longer; Drug clearance was 6% slower; the apparent volume of distribution at steady state was 6% more; Cₘₐₓ was 3% higher; and Tₘₐₓ was 36 minutes sooner. It can be concluded that use of stevioside had positive effects of bioavailability.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A formulation for enhancing bioavailability of vitamin B₉, the formulation comprising:
vitamin B₉; and
a glycoside selected from the group consisting of a diterpene glycoside and a triterpene glycoside.

2. The formulation of claim 1, wherein vitamin B₉ is L-5-methyl-tetrahydrofolate or a salt thereof.

3. (canceled)

4. The formulation of claim 1, wherein the glycoside is a diterpene glycoside.

5. The formulation of claim 4, wherein the diterpene glycoside is selected from the group consisting of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

6. The formulation of claim 5, wherein the diterpene glycoside is selected from the group consisting of stevioside, rebaudioside A, and rebaudioside D.

7. The formulation of claim 6, wherein the diterpene glycoside is stevioside.

8. The formulation of claim 1, wherein the formulation has a ratio of vitamin B9 to the glycoside of 1:1 w/w to 1:30 w/w.

9. (canceled)

10. (canceled)

11. The formulation of claim 1, wherein the formulation has a ratio of vitamin B9 to the glycoside of 1:1 w/w to 1:7 w/w.

12. The formulation of claim 1, wherein vitamin B₉ and said glycoside form a nanoparticle that is about 1 nm to about 10 nm in diameter.

13. (canceled)

14. The formulation of claim 1, wherein the formulation further comprises vitamin B₁₂ and vitamin B₆.

15. The formulation of claim 1, wherein the formulation further comprises vitamin B₁₂ and N-acetylcysteine.

16. The formulation of claim 1, wherein the formulation further comprises vitamin B₁₂.

17. (canceled)

18. A method for making a vitamin B₉ formulation, said method comprising:
admixing vitamin B₉ and a glycoside selected from the group consisting of a diterpene glycoside and a triterpene glycoside in a solvent to form a vitamin B₉ admixture;
heating the vitamin B₉ admixture to form a clear vitamin B₉ admixture; and
removing the solvent from the clear vitamin B₉ admixture to form the formulation of vitamin B₉.

19. The method of claim 18, wherein vitamin B₉ is L-5-methyl-tetrahydrofolate or a salt thereof.

20. (canceled)

21. The method of any claim 18, wherein the glycoside is a diterpene glycoside.

22. The method of claim 21, wherein the diterpene glycoside is selected from the group consisting of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

23. The method of claim 22, wherein the diterpene glycoside is selected from the group consisting of stevioside, rebaudioside A, and rebaudioside D.

24. The method of claim 23, wherein the diterpene glycoside is stevioside.

25. The method of claim 18, wherein the formulation has a ratio of vitamin B₉ to the glycoside of 1:1 w/w to 1:30 w/w.

26. (canceled)

27. (canceled)

28. (canceled)

29. The method of claim 18, wherein the formulation has a ratio of vitamin B₉ to the glycoside of 1:1 w/w to 1:7 w/w.

30. The method of claim 18, wherein the solvent is water.

31. The method of claim 18, wherein the solvent is removed from the clear vitamin B₉ admixture by spray-drying or by lyophilization.

32. (canceled)

33. The method of claim 18, wherein vitamin B₉ and said glycoside form a nanoparticle that is about 1 nm to about 10 nm in diameter.

34. (canceled)

35. A method for increasing the bioavailability of vitamin B₉, the method comprising:
orally administering to a human a formulation comprising vitamin B₉; and a glycoside selected from the group consisting of a diterpene glycoside and a triterpene glycoside; thereby increasing the bioavailability of vitamin B₉ to the human.

36. The method of claim 35, wherein the glycoside is a diterpene glycoside.

37. The method claim 36, wherein the diterpene glycoside is selected from the group consisting of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

38. The method of claim 37, wherein the diterpene glycoside is selected from the group consisting of stevioside, rebaudioside A, and rebaudioside D.

39. The method of claim 38, wherein the diterpene glycoside is stevioside.

40. (canceled)

41. The method of claim 35, wherein the formulation has a ratio of vitamin B₉ to the glycoside of 1:1 w/w to 1:20 w/w.

42. (canceled)

43. The method of claim 35, wherein the formulation has a ratio of vitamin B₉ to the glycoside of 1:1 w/w to 1:7 w/w.

44. (canceled)

45. The method of claim 35, wherein the bioavailability of vitamin B₉ is increased between about 1 to 10 fold as compared to a control containing the same amount of vitamin B₉, but lacking the glycoside.

46. The method of claim 45, wherein the bioavailability of vitamin B₉ is increased between about 2 to 6 fold as compared to the control.

47. The method of claim 35, wherein the formulation further comprises vitamin B₁₂ and vitamin B₆.

48. The method of claim 35, wherein the formulation further comprises vitamin B₁₂ and N-acetylcysteine.

49. The method of claim 35, wherein the formulation further comprises vitamin B₁₂.
